(19) United States
(12) Patent Application Publication
(10) Pub. No.: US 2021/0077309 A1
(43) Pub. Date: Mar. 18, 2021

(54) SANITARY PRODUCT WITH INTEGRATED SENSOR, FABRICATION PLAN THEREFOR AND METHOD THEREOF

(71) Applicant: Abena Holding A/S, Aabenraa (DK)

(72) Inventor: Per Kammeyer, Rødekro (DK)

(21) Appl. No.: 16/959,903

(22) PCT Filed: Jan. 4, 2019
(86) PCT No.: PCT/DK2019/050002

§ 371 Date: Jul. 2, 2020

(30) Foreign Application Priority Data
Jan. 5, 2018 (DK) PA 2018 70007

(51) Int. Cl. A61F 13/42; A61F 13/47; A61F 13/15

(52) U.S. Cl. CPC A61F 13/42; A61F 13/47; A61F 2013/424; A61F 13/15739; A61F 13/15804

(57) ABSTRACT

Aspects of the present disclosure relate to a sanitary product including a back sheet having an outer surface and an inner surface, a core and a front sheet joined with a joining periphery in a sandwich structure with the core fully positioned within the joining periphery. The core positioned between the back sheet and the front sheet with the inner surface of the back sheet facing the core. The sanitary product further including a long direction, a perpendicular short direction and a product periphery, wherein the back sheet includes a printed circuit, a stiffener two through-going incisions.

### FIELD OF THE INVENTION

The present invention relates to a sanitary product comprising a back sheet, having an outer surface and an inner surface, a core and a front sheet joined with a joining periphery in a sandwich structure with the core fully comprised within the joining periphery. The core is arranged between the back sheet and the front sheet with the inner surface of the back sheet facing the core. The sanitary product is configured with a long direction, a perpendicular short direction and a product periphery, wherein the back sheet is configured with a printed circuit on the inner surface, which printed circuit comprises a connector configured with a sensor for fluid detection. The invention relates to the sanitary product, the fabrication plant thereof, the method and a back sheet supply therefore.

### BACKGROUND OF THE INVENTION

A broad variety of sanitary products with incontinence indicators and detection systems exists and are used by people regardless of age from infants to adults. The sanitary products may be absorbent articles and diapers.

Incontinence may have different causes but regardless of the cause for suffering from incontinence, the condition typically involves wearing an absorbent pad or diaper.

For a large group of people suffering from incontinence, checking for wetness may require relying on others. This may be the case for patients in care institutions such as hospitals, and residents in geriatric institutions, care- and nursing homes and similar care centres. An unnoticed wet diaper can become a health risk to the wearer. Diaper rash and skin ulcers are some of the medical conditions that may result from prolonged exposure to wetness.

For both the health risk to the patient and the care providers, processes and procedures for enhanced diaper monitoring are desirable.

To ensure that these patients or residents are adequately cared for, it is necessary for staff to conduct regular checking of the patients suffering from incontinence. The wearers of the sanitary products are often unable to alert staff of the fact that an incontinence event has occurred and, therefore, manual checks are typically carried out on a regular basis. This may place a significant burden on the care providers and causes inconvenience and disruption to the patient.

This need to conduct regular checks of patients for incontinence may be overcome by sanitary products with incontinence indicators and detection systems providing for transmitting a wetness-status of the sanitary product to a remote unit, thereby enabling regular or consistent monitoring of the patient and eliminating the manual checking. The wetness-status may indicate the wetness degree or simply an indicator for whether the fluid is detected or not.

These sanitary products typically include complex sensors with passive and/or active electronic components which are expensive and which may require manual insertion to a diaper.

Achieving an automated process for incorporation of sensor devices into sanitary products, such as diapers, is complicated by the fact that the sanitary products are generally manufactured at high speed on an assembly line using an in-line process. The inline process typically receives the individual pad layers of the sanitary product as a continuous feed from webs, and applies units in the process comprising rotating drums exerting pressure and tension to the various layers. Furthermore, there is generally little lateral or longitudinal precision involved with the in-line process.

The traditional in-line process or the sensors to be incorporated have to be adjusted either by altering the sensors to be sufficiently robust to withstand the forces exerted to them during the fabrication process by the units comprised in the fabrication line, or by altering the fabrication line to incorporate precision control to ensure the reliability of the integrated sensors to work in view of imprecision and forces exerted on the sensors during the process. Alternatively, the fabrication line is adjusted to exert reduced forces, handle more complex sensor units and work with a higher precision in the assembly of the components comprised in the single sanitary products.

WO2017059831A1 discloses a diaper for electronic detection of body fluids for remote monitoring, wherein the sensor is incorporated as a printed circuit in the diaper. The printed circuit comprised in the diaper comprises an electrode, to which a transmitter is connected for transmitting the wetness-status of the diaper. However, the electrode is incorporated in the diaper which means that the transmitter must be connected by conductive pins used to pierce the diaper at the contact part of the electrode. This may require both training and precision in connecting the transmitter to the diaper.

WO 2017/194391A1 also discloses a diaper comprising a wetness detection system. Here two electrodes are bonded to the back sheet or alternatively mechanically anchored to a fabric layer inserted in the diaper. An electronic module can be placed in a pocket included in the diaper and be connected to the two electrodes. The electrodes are mechanically attached to one of the fabric layers of the diaper which may cause stiffness to the diaper and add an additional production step. The electronic module can be connected to the electrodes by sliding the electrodes into the electronics module. However, the fabric layer is only accessible on one fabric layer in a stack of fabric layers which are bonded together in the periphery of the pocket, and thus the accessibility to connecting the electronic module to the electrodes are cumbersome. Attaching the electronic module correctly and to the correct fabric layer may require both training and precision.

### OBJECT OF THE INVENTION

It is an objective to overcome one or more of the before mentioned shortcomings of the prior art. This includes amongst other an objective of providing a sanitary product with an integrated sensor wherein the connection of an electronic module to the integrated sensor is simplified including the accessibility to the connection point. A further objective is to simplify the fabrication of the sanitary product such that the product may be fabricated with high speed in an automated or semi-automated fabrication process.

### DESCRIPTION OF THE INVENTION

An object of the invention may be achieved by a sanitary product comprising a back sheet, having an outer surface and an inner surface, a core and a front sheet joined with a joining periphery in a sandwich structure with the core fully comprised within the joining periphery. The core is arranged between the back sheet and the front sheet with the inner surface of the back sheet facing the core. The sanitary product is configured with a long direction, a perpendicular short direction and a product periphery characterized in that the back sheet is configured with a printed circuit on the inner surface, which printed circuit comprises a connector configured with a connector width and a sensor for humidity detection, preferably fluid detection. The back sheet is further configured with a stiffener for increasing the bending force of the back sheet arranged on the outer surface opposite the connector configured with a stiffener width extending beyond the connector width. The back sheet is furthermore configured with two through-going incisions in the long direction between which incisions the connector width is comprised, the incision going through at least the stiffener and the back sheet, and with one end of each incision ending at the product periphery or at an intersecting cut (144), such that a flap is comprised in the back sheet comprising the connector and part of the stiffener. A sanitary product, wherein the joining periphery encircles a smaller area than the product periphery and is comprised within the product periphery and where the connector and the two through-going incisions in the long direction are outside the joining periphery.

The sensor for humidity detection is preferably for fluid detection in the form of urine. However, it may also be used for detection of humidity in faeces.

The back sheet may comprise a breathable or non-breathable material. The materials may be a textile laminate of polypropylene (PP) and/or polyethylene (PE). The back sheet material may be a nonwoven textile laminate. The back sheet material may be comprised of starch. These are examples of appropriate materials but others may be used.

The back sheet material may have a mass per area in the range 8-80 g/m².

The material chosen for the back sheet may be flexible such that the sanitary product in its entirety is flexible to wear. Furthermore, the back sheet may have a surface making it comfortable to wear against the skin. The sanitary product may not cause any irritations against the skin that can cause rupture, eczema or other skin conditions.

The stiffener may comprise a nonwoven material, polypropylene (PP), polyethylene (PE) and/or other polymer materials which, when applied to the back sheet, increase the bending force of the back sheet. The stiffener may have a mass per area in the range 20-150 g/m².

The stiffener material may be stretchable.

One effect of the embodiment may be that a sanitary product comprising an integrated sensor is achieved, wherein the risk of misalignment of the integrated sensor during use is eliminated.

A further effect may be that the integration of the sensor does not add additional individual items to the sanitary product, such as additional laminate layers or sensor components, which may be misaligned in the joining of the layers comprised in the product. A further advantage is that the printed circuit on the back sheet provides for a robust sensor design in relation to the standard equipment and processes used in standard fabrication processes. The integrated sensor design thus aids for automated fabrication.

Yet a further effect of the embodiment may be that the connector is accessible outside the joint part of the sanitary product. This may be advantageous in regard to not adding a risk of leakage of the sanitary product, when connecting a unit to the connector.

The embodiment provides for a flap being created which is aligned with the product periphery or with a flap created away from the product periphery depending on whether an intersecting cut is provided to intersect the two through-going incisions within the product periphery or if the incisions are intersected by the cut defining the product periphery.

Yet a further effect of the embodiment is that a connector pocket may be formed, comprised by the part of the front sheet and the back sheet outside the joining periphery wherein immediate access to the flap comprising the connector is achieved.

Thus, in immediate continuation hereof, as the stiffener is applied to the back sheet, it may be achieved that the stiffener is not in direct contact with the skin of the wearer of the sanitary product, as the stiffener is arranged such that the part of the front sheet constituting the connector pocket is arranged between the stiffener and the wearers skin when in use.

In a further embodiment of the sanitary product, the stiffener is configured with a stiffener width in the short direction in the range of 20-35 mm, preferably in the range 25-30 mm and a stiffener length in the long direction in the range of 5-20 mm, preferably in the range 10-15 mm.

The size of the stiffener refers to the size of the stiffener comprised in the sanitary product opposite the connector.

Depending on the stiffener material, one effect of this embodiment is that the stiffener is applied to a limited area, thereby, only achieving an increased stiffness of the back sheet in a limited area, which may be advantageous in regard to not influencing on the wear-ability of the product. This includes not influencing on the comfortability of the product against the skin.

The width of the stiffener depends on the width of the connector. The stiffener may be arranged such that the stiffener extends outside the width of the connector. The length of the stiffener should be sufficient to increase the bending force of the back sheet for an easier handling of the flap for connection purposes.

In another aspect, the size of the stiffener is not limiting for the size of the stiffener element applied to the back sheet. The stiffener element may extend in length and width from the size of the stiffener up to the width and/or length of the back sheet. This may have the effect of reinforcing a larger part of the back sheet. This may be advantageous in regard to achieving altered properties of the outer surface of the back sheet and, thereby, the outer side of the sanitary product.

In a further embodiment of the sanitary product, the flap comprised in the back sheet, which comprises the connector and part of the stiffener, is adapted for inserting into and connecting to a transmitter unit.

The transmitter unit may comprise transmission means for transmitting a signal to remote means providing monitoring of wetness-status of the sanitary product. The transmitter unit may transmit a signal to be processed further by the remote means. Alternatively, the transmitter unit may comprise processing means for processing information from the sensor before transmitting information to the remote means.

One effect of this embodiment is that an electrical connection may be provided for, between the connector and the transmitter unit, as the connector of the printed circuit is exposed in the surface of the flap. This may be advantageous in regard to a stable connection. A further effect of this embodiment is that insertion of the flap in the transmitter unit is made easier by reinforcing the back sheet with the stiffener, thereby achieving an effortless and easy handling when connecting the transmitter unit to the sanitary product. The transmitter unit may comprise a slot for the connector to be inserted into. The width of the flap comprising the connector may be adapted accordingly, such that the width of the flap is below the width of the slot, such that the flap may be displaced within the slot. In other words, a slack is provided for the connector in the slot. The displacement may, however, be limited to a distance, which ensures that the connector and the transmitter units are correctly connected. This distance or slack is therefore determined by the connector pins of the two units and/or the distance between the individual connector pins. Furthermore, the slack should be sufficient for easy insertion of the flap into the transmitter unit but limited such that the position of the flap in the transmitter unit is not causing doubt for the user in regard to obtaining a correct connection.

An object of the invention may be achieved by a back sheet supply comprising multiple back sheets, having an outer surface and an inner surface, characterized in that the back sheets are pre-fabricated with a printed circuit on the inner surface comprising a connector with a connector width and a sensor for humidity detection, preferably fluid detection.

The back sheet supply may comprise a continuous web of interconnected back sheets. One effect of this embodiment is achieving a continuous feed of back sheets to a fabrication process, thereby achieving a continuous automatic fabrication process e.g. an in-line process.

An object of the invention may be achieved by a fabrication plant for sanitary products with a production direction adapted for receiving a feed of back sheets comprised in a back sheet supply and for receiving a feed of core and front sheets from a core supply and a—front sheet supply. The fabrication plant comprises a stiffener applicator configured for applying a stiffener element to the back sheets for increasing the bending force of the back sheet on the outer surface opposite the connector configured with a stiffener width extending beyond the connector width. The fabrication plant further comprises an incision device configured for applying through-going incisions in the production direction on either side of the connector, the incision going through the stiffener element and the back sheet. The fabrication plant further comprises a joining device configured to join a back sheet with a core and a front sheet with a joining periphery and with the core fully comprised within the joining periphery. The fabrication plant further comprises a product cutting device for applying a final product cut comprising one or more through-going cuts, defining a product periphery of the sanitary product. A fabrication plant wherein the stiffener applicator and the incision device are arranged before the joining device, wherein the back sheet and the core and the front sheet are arranged with the inner surface facing the core in the joining device, wherein the joining device is configured to join the back sheet with the core and the front sheet such that the connector is outside the joining periphery, and wherein the product cutting device is configured to apply a through-going cut intersecting the through-going incisions in the production direction such that a flap is created in the back sheet comprising the connector and part of the stiffener element.

The fabrication plant may be operated according to fiducials on the back sheet for alignment and thus, for achieving a high precision in the fabrication process and a high precision in the final product. The fiducials may be placed relative to the printed circuit. As an example but not limited to this, a fiducial mark may be placed on a side edge of the back sheet in line with the centre line of the printed circuit. From the fiducial marks, the position of the stiffener and the through-going incisions may be applied accordingly.

For a back sheet supply comprising a continuous web, the fabrication plant may comprise means for exerting a general tension to the back sheets such that the back sheets are extended in length in the production direction. This aids for adjustments in the fabrication without adjusting the position of the devices comprised in the fabrication plant. By exerting a general tension providing extension of the length of the intermediate products in the production direction, the fabrication process may be adjusted by releasing or increasing the tension. Thereby, a misalignment may continuously be accommodated for in the fabrication plant. Alternatively, misalignments may be accommodated for by adjusting the production speed in the fabrication plant. However, for minor misalignments the time response and/or forces required for adjusting the production speed once the fabrication process is running does not in practice provide for continuous adjustments. Such adjustments may instead cause instability due to exceeding adjustments and lengthy time responses (as often seen in controller regulation e.g. PID). Furthermore, the forces required for adjusting the production speed may cause further misalignment and abrasion to the fabrication plant.

One effect of this embodiment is that an automated fabrication process may be achieved for sanitary products with incorporated sensors. The fabrication process may be achieved using standard equipment already used for ordinary sanitary products.

Furthermore, the automated fabrication process may be run as an in-line process, thereby achieving production speeds comparable to those of existing fabrication plants for sanitary products.

The stiffener applicator may comprise a web comprising the stiffener material. The stiffener material is cut in predefined lengths and applied to the back sheets. The application of the stiffeners to the back sheets may be performed by pressure means constituted by rotating drums being displaced relative to each other when the back sheet is displaced in between these pressure means in the production direction. Alternative embodiments of the stiffener applicator may comprise individual stiffeners with a predefined size which are supplied to the back sheets. The stiffeners may be applied by pressure, by heating, by gluing or an alternative joining method or a combination of one or more of these methods. The stiffeners may alternatively be applied as a coating in a liquid form, a spray or a similar way. The property of the stiffener should be such that a potential curing or setting of the stiffener does not provide for any delay in the production speed.

In a further embodiment of the fabrication plant the incision device comprises rotating drums comprising active cutting means for applying through-going incisions in the production direction and pressure means for pressing the back sheet with applied stiffener element against the active cutting means, which means are displaced relative to each other when the back sheet with applied stiffener element is displaced in between the means in the production direction.

One effect of this embodiment is that a standard incision device may be incorporated in the fabrication plant. This device may be advantageous in regard to mechanical stability and providing for a production speed comparable to those of existing fabrication plants for sanitary products. The cutting edges experience wear and should be maintained to ensure sharp incisions. The wear may be highly influenced by the material used for the back sheets and the stiffener.

In yet a further embodiment of the fabrication plant, the incision device comprises a laser.

The incision device may comprise a single laser emitting a beam to be split into two beams for laser cutting through the back sheet with the applied stiffener. The incision device may alternatively comprise two lasers, each emitting a single beam. As a further alternative, the incision device may comprise a plurality of lasers for providing specific ranges of wavelengths and effects adjusted to the materials and thickness of the materials of the back sheet and the stiffener. The length of the incisions is adjusted according to exposure time and the production speed.

The incision device may comprise a continuous wave laser, a pulsed laser or a combination.

One effect of this embodiment is that the laser(s) does/do not comprise mechanical parts which experience wear by physical interaction with the back sheet and the stiffener. Thereby, it will achieve uniform sharpness of the incisions performed in the individual products.

In one aspect, the laser may be a CO₂ laser operating with wavelengths in the spectrum from 9.2-11.4 μm with emitted power in the range of 200-500 W. Alternatively, the laser may be the CO₂ laser operated with emitted power in a range from 200 W up till 4.5 kW.

The wavelength and the emitted power of the laser should be adjusted with respect to the materials to be laser cut to achieve a through-going incision without burning any of the materials.

In yet a further embodiment the fabrication plant is configured to run with a production speed of up to 400 m/min.

One further effect of this embodiment is that an automated fabrication process, for a sanitary product with an integrated sensor, may be run with production speeds comparable to those of existing fabrication plants for sanitary products.

For the various embodiments of the fabrication plant as described above, the fabrication plant comprises a product cutting device providing a final product cut. The embodiment of this device may be similar to any of the embodiments of the incision devices described above. Thus, the incision devices may generally be run at the same production speed.

In the described embodiments of the production plant there is not a single device being the bottleneck providing a limit to the production speed.

For the various embodiments of the fabrication plant as described above, the fabrication process may be achieved using standard equipment already used for ordinary sanitary products. Furthermore, the automated fabrication process may be run as an inline process, thereby achieving production speeds comparable to those of existing fabrication plants for sanitary products. Thus, the described embodiments of the fabrication plant for sanitary products with an integrated sensor is adopted to receive feeds of the individual pad layers of the sanitary product as a continuous feed from webs, and applies units in the process comprising rotating drums exerting pressure and tension to the various layers. Furthermore, the embodiments do not require special precision equipment for running the automated process as an in-line process. Furthermore, the devices comprised in the fabrication plant is not adjusted to exert reduced forces, handle more complex sensor units or work with a higher precision in the assembly of the components comprised in the single sanitary products, compared to commonly used devices.

An object of the invention may be achieved by a method for producing a sanitary product comprising acts of providing a back sheet configured with an inner surface and an outer surface, wherein the back sheets are pre-fabricated with a printed circuit on the inner surface, comprising a connector configured with a connector width and a sensor for humidity detection, preferably fluid detection, and applying a stiffener element to the back sheet for increasing the bending force of the back sheet on the outer surface opposite the connector configured with a stiffener width extending beyond the connector width. The method further comprises acts of performing two through-going incisions in the production direction on either side of the connector going through at least the stiffener element and the back sheet (102), and providing a core and a front sheet. Furthermore, the method comprises acts of joining the back sheet and the core and the front sheet with the inner surface of the back sheet facing the core to provide a joining periphery with the core fully comprised within the joining periphery, and performing a final product cut comprising one or more through-going cuts defining a product periphery of the sanitary product. A method, wherein the connector is comprised outside the joining periphery and an intersecting cut (144) which intersects the through-going incisions in the production direction, such that a flap is created in the back sheet comprising the connector and part of the stiffener element.

One effect of this method may be that the integration of the sensor only requires acts which are commonly known acts from fabrication methods in other industries, but acts which may be performed in an automated fabrication method and in-line productions. This may be advantageous in regard to achieving a fabrication method which does not require any intermediate acts where any intermediate products are on hold for cure, drying or comparable tasks.

The effects and advantages of the method are in line with those already described in connection with the embodiments of the sanitary product, the fabrication plant, the fabrication method and the back sheet supply.

Thus, the further effects and advantages are amongst others as listed below.

A further effect of the method is that it provides for a sanitary product comprising a connector which is accessible outside the joined part of the sanitary product. This may be advantageous in regard to not adding a risk of leakage of the sanitary product when connecting a transmitter unit to the connector.

Yet a further effect of the method is that a connector pocket may be formed, comprised by part of the front sheet and the back sheet outside the joining periphery, wherein immediate access to the flap comprising the connector is achieved.

Thus, in immediate continuation hereof, as the stiffener is applied to the back sheet, it may be achieved that the stiffener is not in direct contact with the skin of the wearer of the sanitary product, as the stiffener is arranged in a way where the part of the front sheet constituting the connector pocket is arranged between the stiffener and the user's skin when in use.

The method further incorporates a number of precautionary conditions for mitigating the risk of misalignment and/or imprecisions during the fabrication.

These precautionary conditions include the stiffener element and the two through-going incisions. These may all extend in length in the long direction of the product, which is also the production direction. They may extend in length so the final product cut intersects the through-going incisions and the stiffener element. Furthermore, the stiffener element and the stiffener have a width extending beyond the connector width. These conditions in combination ensure that a flap comprising the connector may be achieved, which is reinforced in the entire area comprised in the flap. This may, as previously described, be advantageous in regard to achieving:
a stable connection between the connector and the transmitter unit
easy insertion of the flap in the transmitter unit
easy handling for connecting the transmitter unit to the sanitary product.

In a further embodiment the method the back sheets are provided as a continuous web comprising a further act of exerting a general tension to the intermediate products in the production direction, such that the back sheets are extended in length in the long direction.

One further effect of this embodiment is that a continuous adjustment in the fabrication direction is achieved without continuously adjusting the position of the devices comprised in the fabrication plant and/or adjusting the production speed.

Thus, this is may be further advantageous in regard to achieving that a misalignment may continuously be accommodated for in the fabrication plant.

Alternatively, misalignments may be accommodated for by adjusting the production speed in the fabrication plant. However, for minor misalignment the time response and/or forces required for adjusting the production speed, once the fabrication process is running does not in practice provide for continuous adjustments. Such adjustment may instead cause instability due to exceed adjustments and lengthy time responses (as often seen in controller regulation e.g. PID). Furthermore, the forces required for adjusting the production speed may cause further misalignment and abrasion to the fabrication plant.

Thus, the method provides for an automated fabrication process which may be run as an in-line process, thereby achieving production speeds comparable to those of existing fabrication plants for sanitary products. The described embodiments of the method for producing sanitary products with integrated sensors is adopted to provide continuous feeds of the components comprised in the sanitary product from webs, and applies acts in the process exerting pressure and tension to the various layers. The embodiments do not require special precision equipment for running the automated process as an in-line process. Furthermore, the acts of the method is not adjusted to exert reduced forces, handle more complex sensor units or work with a higher precision in the assembly of the components comprised in the single sanitary products, compared to commonly used fabrication methods.

An object of the invention may be achieved by a sanitary product obtained by the method for producing a sanitary product.

The effects and advantages of the method are in line with those already described in connection with the embodiments of the sanitary product, the fabrication plant, the fabrication method and the back sheet supply.

In one aspect an object of the invention may be achieved by an alternative embodiment of the sanitary product. The alternative embodiment of the sanitary product is a comparable product to the other embodiments of the sanitary product, differing in that the connector on the inner surface of the back sheet is displaced in the long direction of the product away from the product periphery towards the centre of the back sheet. The stiffener element is arranged opposite the connector on the outer surface of the back sheet and extends in width beyond the connector width. Two through-going incisions going through at least the back sheet and the stiffener element are provided on either side of the connector. However, a third incision is provided going through the back sheet and the stiffener element in the short direction of the product intersecting the two through-going incisions. The third incision is provided at the end of the connector or in the close vicinity thereof, in the end of the connector not being connected to the sensor. The alternative embodiment of the sanitary product, may thereby be provided with a flap in the back sheet, which is comprised within the connection pocket but not being a part of the product periphery. Comparable to the other embodiments of the sanitary product, the flap comprising the connector is reinforced such that the bending force of the back sheet is increased.

### DESCRIPTION OF THE DRAWING

FIG. 1 illustrates one embodiment of the sanitary product with indications of the core and printed circuit.

FIG. 2 illustrates one embodiment of the sanitary product inserting into and connecting to a transmitter unit.

FIG. 3 illustrates one embodiment of the back sheet supply.

FIG. 4 illustrates one embodiment of the fabrication plant in perspective view.

FIG. 5 illustrates one embodiment of the fabrication plant in side view.

FIG. 6 illustrates one embodiment of the incision device.

FIG. 7 illustrates an alternative embodiment of the fabrication plant in perspective view.

FIG. 8 illustrates an alternative embodiment of the incision device.

FIG. 9 illustrates one embodiment of the method for producing a sanitary product.

FIG. 10 illustrates one embodiment of an intermediate product of an alternative sanitary product.

### DETAILED DESCRIPTION OF THE INVENTION

| No | Item |
|---|---|
| 10 | transmitter unit |
| 20 | connector pocket |
| 100 | sanitary product |
| 101 | alternative sanitary product |
| 102 | back sheet |
| 104 | core |
| 106 | front sheet |
| 108 | joining periphery |
| 110 | long direction |
| 112 | short direction |
| 114 | product periphery |
| 120 | inner surface |
| 122 | printed circuit |
| 124 | connector |
| 126 | connector width |
| 128 | sensor |
| 130 | outer surface |
| 132 | stiffener |
| 136 | stiffener width |
| 138 | stiffener length |
| 140 | through-going incision |
| 142 | third incision |
| 150 | flap |
| 200 | back sheet supply |
| 210A | core supply |
| 210B | front sheet supply |
| 300 | fabrication plant |
| 302 | production direction |
| 310 | stiffener applicator |
| 312 | stiffener element |
| 314 | stiffener element length |
| 320 | incision device |
| 322 | active cutting means |
| 324 | pressure means |
| 326 | laser |
| 330 | joining device |
| 340 | product cutting device |
| 342 | final product cut |
| 344 | through-going cuts |
| 360 | production speed |
| 400 | method |
| 410 | providing |
| 420 | applying |
| 430 | performing |
| 440 | joining |

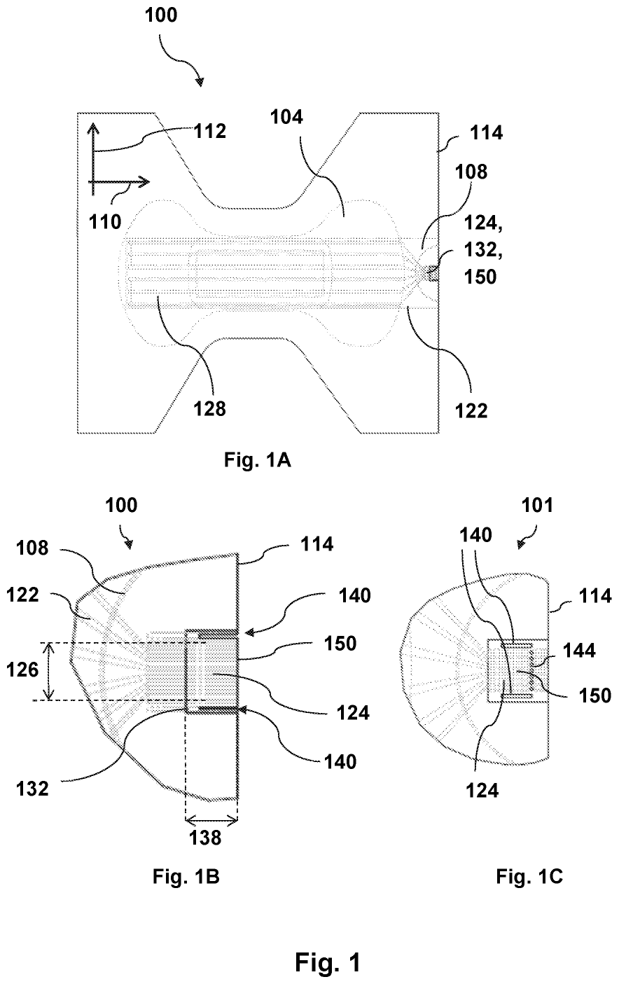

FIG. 1 illustrates one embodiment of the sanitary product 100 with indications of the core 104 and printed circuit 122. FIG. 1A illustrates the entire product 100 and FIG. 1B illustrates a detailed section of the product 100 of the part comprising the connector 124. FIG. 1C illustrates a detailed section of the alternative sanitary product 101 of the part comprising the connector 124.

The illustrated sanitary product 100 in FIG. 1A comprises a core 104 comprised between a back sheet and a front sheet (not explicitly illustrated). A printed circuit 122 is arranged on the inner surface of the back sheet facing the core 104. The printed circuit 122 comprises a sensor 128 for fluid detection and a connector 124. The back sheet, the core 104 and the front sheet are joined with a joining periphery 108 within which the core 104 is fully comprised. The sanitary product 100 may be defined by a long direction 110 and a perpendicular short direction 112. The sanitary product 100 may be further defined by a product periphery 114, being the outer periphery of the product 100. The joining periphery 108 encircles a smaller area than the product periphery 108, which is illustrated on the right hand side of FIG. 1A. Here, the joining periphery 108 is illustrated with a curvature extending inwards toward the core 104 leaving the part of the product comprising the connector 124, the stiffener 132 and the incisions 140 in the stiffener 132 and the back sheet outside of the joining periphery 108. This part is illustrated in more detail in FIG. 1B. The part of the product comprised outside the joining periphery 108 and within the product periphery 114 provides for a pocket (connector pocket 20, not illustrated here) in the product constituted of the front sheet and the back sheet.

A part of the printed circuit 122 comprising the connector 124 is illustrated in FIG. 1B. The stiffener 132 having a stiffener length 138 is arranged opposite the connector 124 on the back sheet and extends in width (in the short direction) beyond the connector width 126. Two through-going incisions 140 going through the back sheet and the stiffener 132 is provided on either side of the connector 124 ending at the product periphery 114, thereby providing for a flap to be enabled in the back sheet. The flap comprising the connector is reinforced such that the bending force of the back sheet is increased.

The stiffener 132 is preferably arranged so that it extends in length in one end to reach the product periphery 114 in the end comprising the non-joined area comprising the connector 124.

In FIG. 1C the two through-going incisions 140 going through the back sheet and the stiffener 132 is also provided on either side of the connector 124, however, the incisions 140 do not extend to the product periphery 114. Instead an intersecting cut 144, here illustrated as a perforation, intersects the two through-going incisions 140 leaving the connector to be connected to the sensor, such that the flap 150 is created away from the product periphery 114.

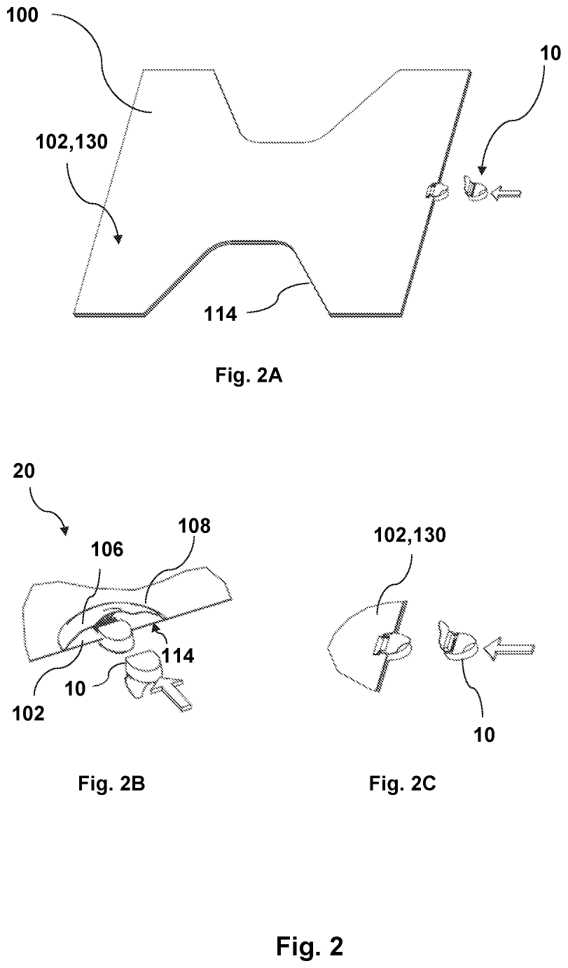

FIG. 2 illustrates one embodiment of the sanitary product 100 where a part of the product is inserted into and connected to a transmitter unit 10 at the product periphery 114. FIG. 2A illustrates the full sanitary product 100 as seen from the outer surface 130 of the back sheet 102. The transmitter unit 10 is connected to the reinforced part of the back sheet 102 comprising the connector.

FIGS. 2B and 2C illustrates the connection of the transmitter unit 10 with the back sheet 102 in more detail, where FIG. 2B illustrates the connection as seen from the top sheet 106, which is the skin side of the sanitary product 100. FIG. 2C illustrates the connection as seen from the outer surface 130 of the back sheet 102, which is the outer side of the sanitary product 100. FIG. 2B further illustrates the connector pocket 20 in the product 100 constituting the front sheet 106 and the back sheet 102, which connector pocket 20 arises from the part of the product 100 comprised outside the joining periphery 108 and within the product periphery 114.

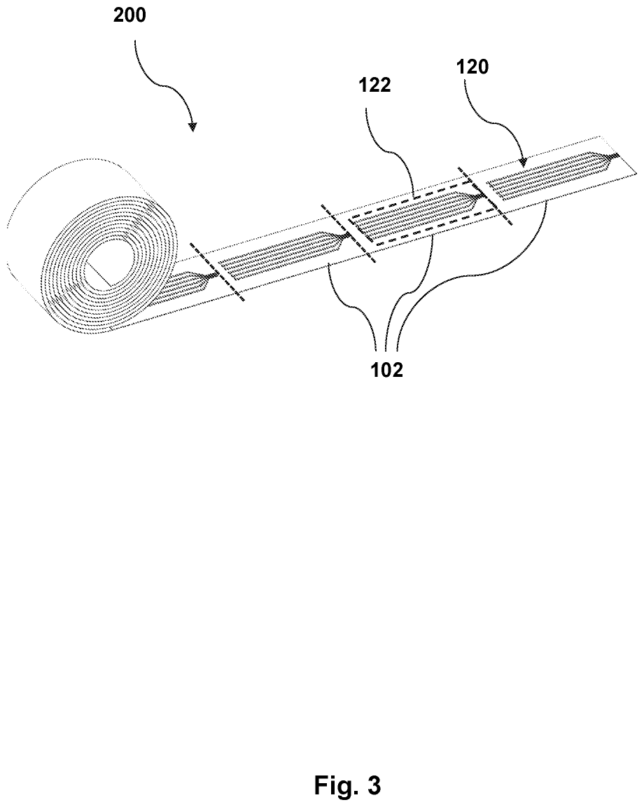

FIG. 3 illustrates one embodiment of the back sheet supply 200. Here the back sheet supply 200 comprises a continuous web with pre-applied printed circuits 122. Thus, the back sheet supply 200 comprises a feed of interconnected back sheets 102 illustrated by the dotted lines, which back sheets 102 comprises printed circuits 122 on the inner surface 120.

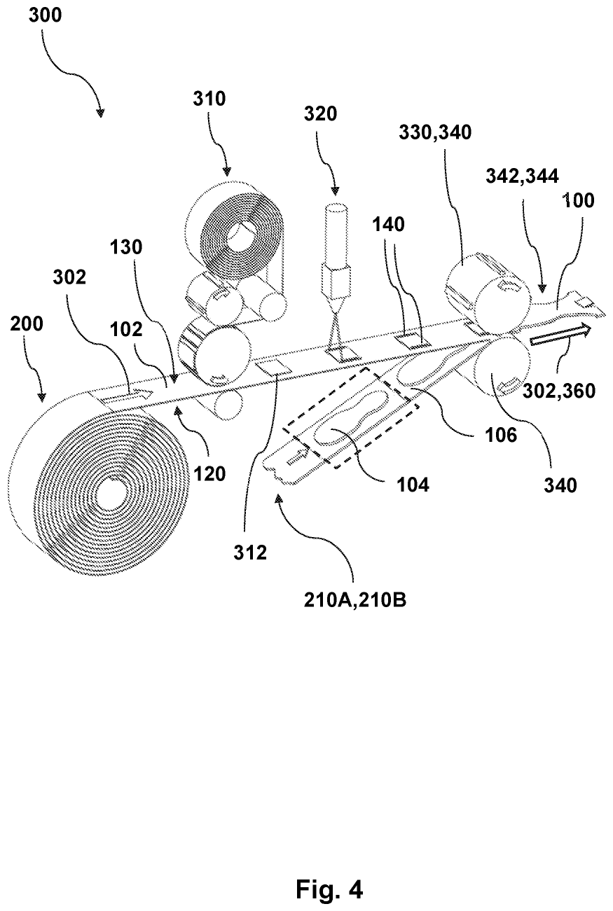

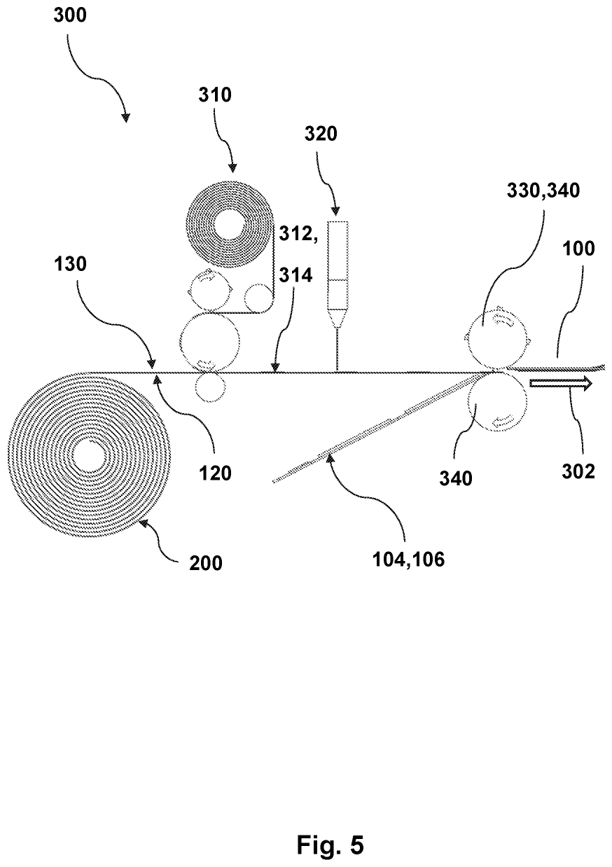

FIGS. 4 and 5 illustrates one embodiment of a fabrication plant 300 for sanitary products with a production direction 302 and a production speed 360. The fabrication plant 300 is illustrated in perspective view in FIG. 4 and in side view in FIG. 5. The production plant 300 provides for in-line production of the sanitary products. A feed of back sheets 102 from a back sheet supply 200 is provided to the fabrication plant 300. Here, the back sheet supply 200 is illustrated as a continuous web. Thus, the feed of back sheets 102 is provided from a back sheet supply 200 comprising interconnected back sheets 102 pre-applied with printed circuits 122 (not illustrated here) on the inner surface 120.

The fabrication plant further comprises a stiffener applicator 310 configured for applying a stiffener element 312 to the back sheets 102 for increasing the bending force of the back sheet 102. The stiffener elements 312 are applied on the outer surface 130 of the back sheets 102 opposite the connector of the printed circuits, which are not illustrated here.

The stiffener element 312 is the same size or larger than the stiffener comprised in the sanitary product. The size of the stiffener element 312 and the stiffener may differ in length. The reason for this is to mitigate the risk of misalignments in the fabrication. By having a stiffener element 312 extending in length beyond the stiffener length, the stiffener element may be arranged such that a part of the element may extend beyond the product periphery 114 in the end comprising the non-joined area comprising the connector 124. When the final product cut 342 is provided in the area comprising the stiffener element 312, it may thereby be ensured that the stiffener in the sanitary product is arranged so it is aligned with the product periphery 114 in the end comprising the non-joined area comprising the connector 124.

In one aspect where the back sheet supply 200 comprises a continuous web of connected back sheets 102, the stiffener element 312 may overlap from one back sheet 102 to the next and thus, provide the final product cut in the area comprising the stiffener element 312. It is thereby ensured that the stiffener in the sanitary product is arranged in a manner where it is aligned with the product periphery 114 in the end comprising the non-joined area comprising the connector 124.

The stiffener may extend in length to the product periphery of the connector pocket. The stiffener element may extend beyond the product periphery, thereby extending in length beyond the stiffener comprised in the final sanitary product.

The embodiment illustrated here of the stiffener applicator 310 comprises a web comprising the stiffener material. The stiffener material is cut in predefined lengths and applied to the back sheets. The application of the stiffeners to the back sheets 102 is performed by pressure means being displaced relative to each other when the back sheet 102 is displaced between these pressure means in the production direction.

The stiffener material may be stretchable.

Other embodiments of the stiffener applicator 310 may comprise individual stiffeners with a pre-defined size, which are supplied to the back sheets 102. The stiffeners may be applied by pressure, by heating, by gluing or an alternative joining method or a combination of one or more of these methods. The stiffeners may alternatively be applied as a coating in a liquid form, a spray or a similar way. The property of the stiffener should be such that a potential curing or setting of the stiffener does not provide for any delay in the production speed.

The fabrication plant 300 further comprises an incision device 320 configured for providing through-going incisions 140 in the production direction 302. The incision may be supplied on either side of the connector 124 (not illustrated here). The incisions may be provided such that they penetrate the stiffener element 312 and the back sheet 102.

A feed of cores 104 and front sheets 106 from a core supply 210A and a front sheet supply 210B comprising cores (104) and front sheets 106 is provided to the fabrication plant 300. Here, the core and front sheet supplies 210A,210B are illustrated as a continuous web. The cores 104 and front sheets 106 may be fed individually into the fabrication plant 300, which may be adapted accordingly.

Furthermore, the fabrication plant 300 may comprise a joining device 330 configured to join a back sheet 102 with a core 104 and a front sheet 106, and a product cutting device 340 for applying a final product cut 342 as one or more through-going cut 344 defining a product periphery of the sanitary product 100.

Production direction 302 refers to the directions of the back sheets 102 through the fabrication plant, and the later direction of the final product, the sanitary product 100, as output from the fabrication plant 300.

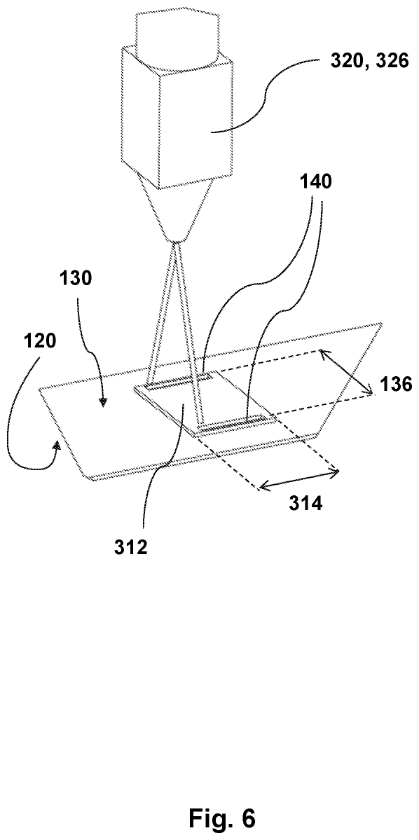

FIG. 6 illustrates one embodiment of the incision device 320. The incision device comprises a laser 326 emitting two beams to the back sheet with the applied stiffener element 312. The stiffener 132 with a stiffener element length 314 and a stiffener width 136 is applied to the outer surface 130 of the back sheet. The beams are emitted for a time duration, which is correlated with the production speed, thereby obtaining incisions 140 of a given length. Two through-going incisions 140 are provided in the production direction 302 (not illustrated here), so the connector width is comprised between the incisions 140. The incisions are through-going incisions 140, penetrating both the stiffener element 312 and the back sheet.

In one aspect the incisions may be limited to be within the length of the stiffener element 314 to avoid any burning of the back sheet if the laser beams are applied solely to the back sheet. Such burning may arise because the effect emitted by the laser is adjusted to penetrate a combination of materials with a thickness larger than the thickness of the back sheet material alone.

In the illustrated embodiment the incisions are provided in the direction from the stiffener element 312 to the back sheet with the inner surface 120 of the back sheet facing away from the laser 326. In an alternative embodiment, the incision may be provided in the opposite direction from the back sheet to the stiffener element 312 with the inner surface 120 of the back sheet facing towards the laser 326.

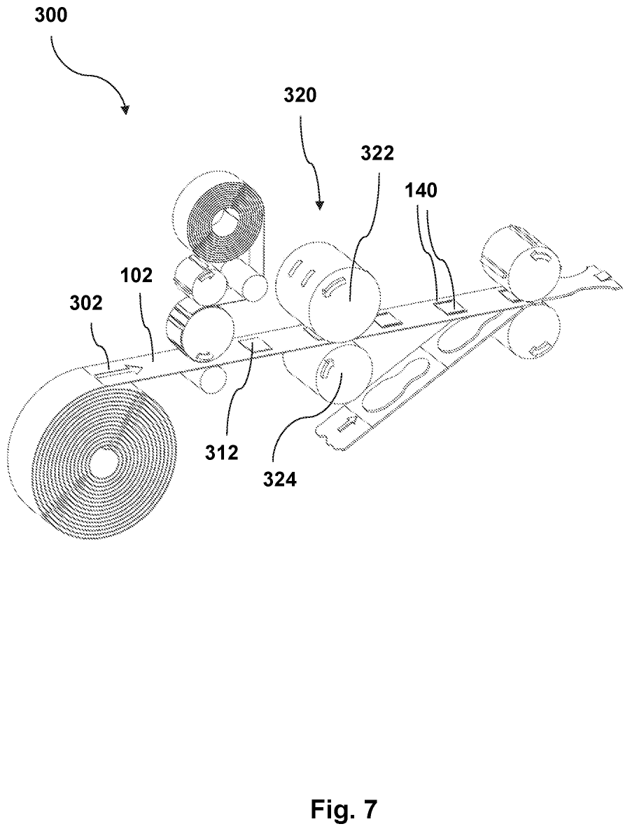

FIG. 7 illustrates an alternative embodiment of the fabrication plant 300 for sanitary products with a production direction 302 in perspective view. Similar to the embodiment illustrated in FIGS. 4 and 5 the production plant 300 provides for in-line production of the sanitary products. The embodiment in FIG. 7 is different from the embodiment illustrated in FIGS. 4 and 5 in that the incision device 320 is a mechanical device comprising rotating drums and cutting edges. The incision device 320 comprises active cutting means 322 for applying through-going incisions 140 in the production direction 302 and pressure means 324 for pressing the back sheet 102 with applied stiffener element 312 against the active cutting means 322 which means 322,324 are displaced relative to each other when the back sheet 102 with applied stiffener element 312 is displaced in between the means 322,324 in the production direction 302.

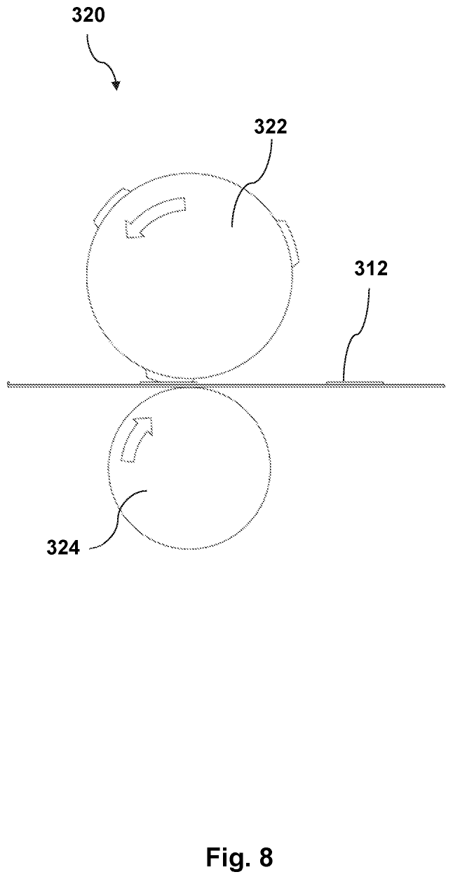

The incision device is illustrated in more detail in FIG. 8 in a side view illustrating the active cutting means 322 for applying the through-going incisions 140 and the pressure means 324 for pressing the back sheet 102 with an applied stiffener element 312 against the active cutting means 322. The displacement of the cutting and pressure means 322,324 are illustrated by the arrows showing a rotational motion of both means for achieving the means to be displaced relative to each other when the back sheet 102 with applied stiffener 132 is displaced or moved in a linear motion in between the means 322,324. The incisions are through-going incisions 140 penetrating both the stiffener element 312 and the back sheet. In the illustrated embodiment, the incisions are provided in the direction from the stiffener element 312 to the back sheet. In an alternative embodiment the incision may be provided in the opposite direction from the back sheet to the stiffener element 312.

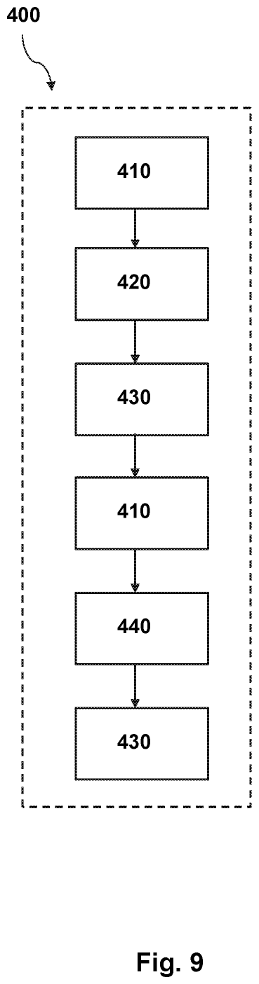

FIG. 9 illustrates one embodiment of the method 400 for producing a sanitary product. The illustrated embodiment comprises the act of providing 410 a back sheet. The back sheet may be configured with an inner surface and an outer surface, wherein the back sheets are pre-fabricated with a printed circuit on the inner surface comprising a connector configured with a connector width and a sensor for fluid detection. The method further comprises an act of applying 420 a stiffener element to the back sheet. The stiffener element is applied to increase the bending force of the back sheet on the outer surface opposite the connector configured with a stiffener width extending beyond the connector width. The method further comprises an act of performing 430 two through-going incisions in the production direction on either side of the connector penetrating the stiffener element and the back sheet. The method further comprises the act of providing 410 a core and a front sheet. The method further comprises an act of joining 440 the back sheet and the core and the front sheet with the inner surface of the back sheet facing the core to provide a joining periphery with the core fully comprised within the joining periphery. The method furthermore comprises an act of performing 430 a final product cut defining a product periphery of the sanitary product. The final product cut is performed such that the connector is comprised outside the joining periphery. The final product cuts may be provided such that they intersect the through-going incisions in the production direction for a flap to be created in the back sheet, which flap comprises the connector and part of the stiffener element.

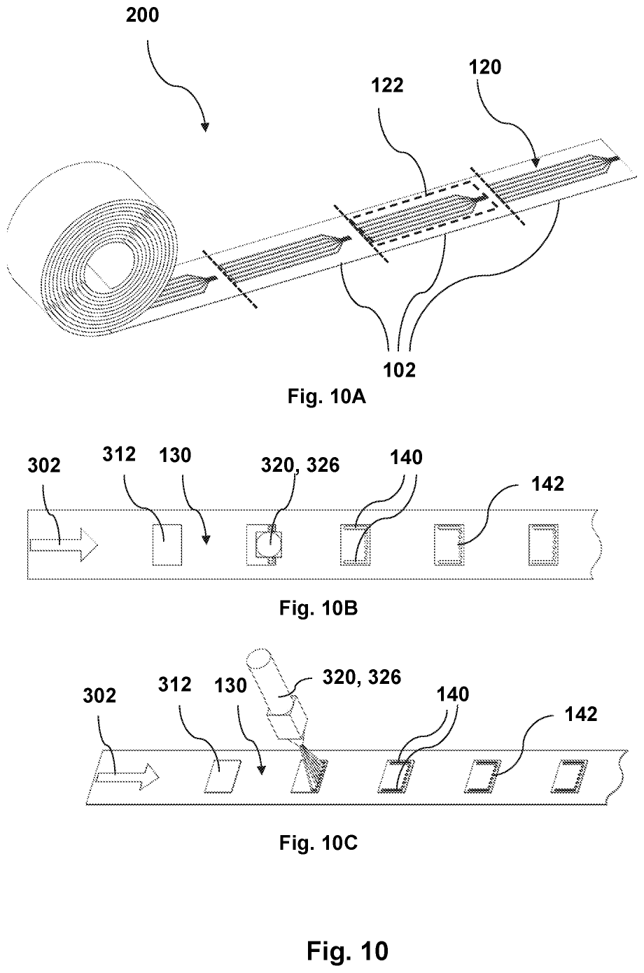

FIG. 10 illustrates an alternative embodiment of an intermediate product in the fabrication process resulting in an alternative embodiment of the sanitary product. The resulting alternative sanitary product 101 is a comparable product to the other embodiments of the sanitary product 100, differing in that the connector on the inner surface 120 of the back sheet 102 is displaced in the long direction of the product away from the product periphery towards the centre of the back sheet 102. The stiffener element 312 is arranged opposite the connector on the outer surface 130 of the back sheet 102 and extends in width (in the short direction) beyond the connector width. Two through-going incisions 140 going through the back sheet 102 and the stiffener element 312 is provided on either side of the connector. However, a third incision 142 is provided going through the back sheet 102 and the stiffener element 312 in the short direction of the product intersecting the two through-going incisions 140. The third incision 142 is provided at the end of the connector or in the close vicinity thereof, in the end of the connector not being connected to the sensor. The alternative embodiment of the sanitary product, may thereby be provided with a flap in the back sheet, which is comprised within the connection pocket but not being a part of the product periphery. Comparable to the other embodiments of the sanitary product, the flap comprising the connector is reinforced such that the bending force of the back sheet is increased.

A back sheet supply 200 comparable to that previously described is applied, comprising a continuous web with pre-applied printed circuits 122. Thus, the back sheet supply 200 comprises a feed of interconnected back sheets 102 as illustrated by the dotted lines in FIG. 10A. Each back sheet 102 comprises a printed circuit 122 on the inner surface 120. However, as illustrated in FIG. 10A the printed circuit 122 on each back sheet 102 is arranged such that the connector is displaced away from the outer periphery of each back sheet 102 towards the centre of the back sheet 102.

FIGS. 10B and 10C illustrate the alternative embodiment of an intermediate product in the fabrication process. FIG. 10B illustrates a top view and FIG. 10C illustrates a perspective view. Stiffener elements 312 are applied to the outer surface 130 of each back sheet comprised in the continuous web. The stiffener elements 312 are applied for increasing the bending force of the back sheets 102. The stiffener elements 312 are applied on the outer surface 130 of the back sheets 102 opposite the connector of the printed circuits.

The stiffener element 312 is the same size or larger than the stiffener comprised in the sanitary product. The size of the stiffener element 312 and the stiffener may differ in length. The reason for this is to mitigate the risk of misalignments in the fabrication process.

The fabrication plant further comprises an incision device 320, here illustrated in an embodiment, where the incision device 320 is a laser 326. The incision device 320 is configured for providing two through-going incisions 140 in the production direction 302 to each back sheet. The incision device 320 may be further configured for providing a third incision 142 to each back sheet intersecting the two through-going incisions 140. The incision may be supplied on either side of the connector 124 (not illustrated here). The incisions may be provided such that they penetrate the stiffener element 312 and the back sheet 102. The third incision 142 is provided going through the back sheet 102 and the stiffener element 312. The third incision 142 is provided at the end of the connector or in the close vicinity thereof, in the end of the connector not being connected to the sensor.

It may thereby be ensured that the stiffener in the sanitary product is arranged such that it is aligned with the flap defined by the two through-going incisions 140 and the third incision 142 intersecting the two through-going incisions 140. The third incision 142 may be a perforation rather than an incision, which may entail that the flap may be fully released once the product is being used.

What is claimed is:

1. A sanitary product comprising:
a back sheet having an outer surface and an inner surface;
a core;
a front sheet joined with a joining periphery in a sandwich structure with the core fully positioned within the joining periphery and between the back sheet and the front sheet, the inner surface of the back sheet facing the core;
a long direction;
a perpendicular short direction;
a product periphery; and
wherein the back sheet further includes
a printed circuit on the inner surface of the back sheet, the printed circuit including a connector configured with a connector width and a sensor configured and arranged for humidity detection;
a stiffener configured and arranged to increase the bending force of the back sheet on the outer surface opposite the connector, and the connector having a stiffener width extending beyond the connector width; and
two through-going incisions in the long direction, between which the connector width is defined, the incisions going through at least the stiffener and the back sheet, and with one end of each of the two incisions ending at the product periphery or at an intersecting cut forming a flap in the back sheet including the connector and part of the stiffener;
wherein the joining periphery encircles a smaller area than the product periphery, the joining periphery is within the product periphery, and the connector and the two through-going incisions in the long direction are outside the joining periphery.

2. The sanitary product according to claim 1, wherein the stiffener includes a stiffener width, in the short direction, in the range of 20-35 mm, and a stiffener length in the long direction in the range of 5-20 mm.

3. The sanitary product according to claim 1, wherein the flap is configured and arranged for inserting into and connecting to a transmitter unit.

4. The sanitary product of claim 1, further including a back sheet supply including multiple back sheets, wherein said multiple back sheets have an outer surface and an inner surface, the back sheets are configured and arranged to be pre-fabricated with the printed circuit.

5. A system for manufacturing sanitary products in a production direction, the system comprising:
a back sheet supply including multiple back sheets, the multiple back sheets including an outer surface and an inner surface, and the back sheets include a printed circuit on the inner surface, the printed circuit including a connector with a connector width and a sensor configured and arranged for humidity detection;
a core supply;
a front sheet supply;
the production direction configured and arranged to
receive a feed of back sheets from the back sheet supply, and
receive a feed of cores and front sheets from the core supply and the front sheet supply;
a stiffener applicator configured and arranged for applying a stiffener element to the back sheets, and thereby increasing a bending force of the back sheet on the outer surface opposite the connector, the stiffener element having a stiffener width extending beyond the connector width;
an incision device configured and arranged for applying through-going incisions in the production direction on either side of the connector, the incision extending through the stiffener element and the back sheet;
a joining device configured and arranged to join a back sheet with a core, and a front sheet with a joining periphery, the core fully enclosed within the joining periphery; and
a product cutting device configured and arranged for applying a final product cut including one or more through-going cuts, the product cut defining a product periphery of the sanitary product;
wherein the stiffener applicator and the incision device are arranged before the joining device, the back sheet the core and the front sheet are arranged with the inner surface facing the core in the joining device, the joining device is further configured and arranged to join the back sheet with the core and the front sheet such that the connector (124) is outside the joining periphery, and the product cutting device is further configured and arranged to apply a through-going cut intersecting the through-going incisions in the production direction such that a flap is created in the back sheet including the connector (124) and part of the stiffener element (314).

6. The system of claim 5, wherein the incision device includes
rotating drums with active cutting means configured and arranged to apply through-going incisions in the production direction and
pressure means (324) for pressing the back sheet (102) with applied stiffener element (312) against the active cutting means (322); and
wherein the active cutting means and the pressure means are configured and arranged to be displaced relative to each other when the back sheet, with applied stiffener element, is displaced in between the active cutting means and the pressure means in the production direction.

7. The system of claim 5, wherein the incision device is a laser.

8. The system of claim 5, wherein the system is configured and arranged to run with a production speed of up to 400 m/min.

9. A method for producing a sanitary product comprising acts of:
providing a back sheet with an inner surface and an outer surface, wherein the back sheet is pre-fabricated with a printed circuit on the inner surface, the printed circuit including a connector with a connector width and a sensor for humidity detection;
applying (420) a stiffener element to the back sheet for increasing the bending force of the back sheet on the outer surface opposite the connector with a stiffener width extending beyond the connector width;
performing two through-going incisions in a production direction on either side of the connector going through at least the stiffener element and the back sheet;
providing a core and a front sheet (106);
joining (440) the back sheet, the core and the front sheet, with the inner surface of the back sheet facing the core, thereby forming a joining periphery with the core fully enveloped within the joining periphery; and
performing a final product cut including one or more through-going cuts defining a product periphery of the sanitary product;
wherein the connector is positioned outside the joining periphery and an intersecting cut, the intersecting cut intersects the through-going incisions in the production direction creating a flap in the back sheet including the connector and part of the stiffener element.

10. The method according to claim 9, wherein the step of providing a back sheet further includes providing a continuous web by exerting a general tension to intermediate products in the production direction, such that the back sheet is extended in length in a long direction.

11. (canceled)

12. The sanitary product of claim 1, wherein the sensor is further configured and arranged for fluid detection.

13. The method according to claim 9, wherein the sensor detects fluid.

14. The sanitary product according to claim 1, wherein the stiffener includes a stiffener width, in the short direction, in the range of 25-30 mm, and a stiffener length, in the long direction, in the range of 10-15 mm.
